(19) United States
(12) Patent Application Publication
(10) Pub. No.: US 2017/0128253 A1
(43) Pub. Date: May 11, 2017

(54) DEVICES AND METHODS FOR SUPPORTING THE HEAD AND NECK

(71) Applicant: WALTER WILLIAMS, Conyers, GA (US)

(72) Inventor: WALTER WILLIAMS, Conyers, GA (US)

(21) Appl. No.: 15/295,213

(22) Filed: Oct. 17, 2016

Related U.S. Application Data
(60) Provisional application No. 62/252,910, filed on Nov. 9, 2015.

(51) Int. Cl. A61F 5/058; A61F 5/37; A61F 5/02

(52) U.S. Cl. CPC A61F 5/05883; A61F 5/026; A61F 5/3707; A61F 5/05825

(57) ABSTRACT

Disclosed herein are devices and methods for supporting the head and neck of a wearer. The head and neck support device can have a head cover and a harness. The head cover can have a head-receiving component and a pair of neck straps. The harness can have a chest portion, an opposed back portion, and first and second attachment zones for securing the neck straps to the harness.

### CROSS-REFERENCE TO RELATED APPLICATIONS

This application claims priority to and the benefit of U.S. Provisional Patent Application Ser. No. 62/252,910, filed Nov. 9, 2015, which application is hereby incorporated by reference herein in its entirety.

### FIELD

This application relates generally to devices and methods for posture support and spinal alignment. More specifically, devices and methods are provided for supporting the head and neck of a wearer in an upright position.

### BACKGROUND

Various devices have been used to support the head or neck of wearer in a desired position. Historically, such devices are used by individuals recovering from whiplash, an auto accident injury, a sport injury, or a fall. These devices are designed using stiff plastic and bulky materials, including a series of straps, buckles and belts. More recently, devices with an elastic strap and a mechanically interconnecting hat have been developed. An example of such a device is described in U.S. Patent Application Publication No. 2012/0260925, which is incorporated herein by reference. However, there are at least two major disadvantages associated with these devices. First, the strap has a tendency to drop from the forehead position when the body is in motion, losing resistance and causing the head to fall from an upright position. In addition, the strap crosses behind the wearer's head and rear torso such that if the strap is crossed incorrectly, application of tension can be difficult. In such instances, the wearer's head can be lifted in an undesirable position, causing increased pain, stiffness, and/or numbness in the neck, shoulders, and/or arms of the wearer.

Accordingly, there remains a need for devices and methods for comfortably supporting the head and neck of a wearer in an upright, neutral position. There is a further need for providing such head and neck support using devices that employ light-weight, nonmetallic pieces that do not cross behind the wearer's neck. These needs and other needs may be satisfied by the various aspects of the present disclosure.

### SUMMARY

Disclosed herein, in one aspect, is a head and neck support device for supporting a head and neck of a wearer. In one aspect, the head and neck support device can comprise a head cover. In this aspect, the head cover can comprise a head-receiving component that has an interior surface and an exterior surface. In a further aspect, the head-receiving component can have a front portion and an opposed rear portion. In one aspect, it is contemplated that the interior surface of the head-receiving portion can be configured to receive at least a portion of the head of a wearer. In another aspect, the head cover can comprise a pair of neck straps. In this aspect, each neck strap can have a first end secured to the rear portion and an opposed second end. In one aspect, it is contemplated that each neck strap can extend downwardly from the head-receiving component relative to a vertical axis (or another axis that, in use, is parallel or generally parallel to the spine of the wearer).

In another aspect, the head and neck support device can comprise a harness. In one aspect, the harness can have a chest portion and an opposed back portion. In another aspect, the chest portion can be configured to surround at least a portion of the chest of the wearer. In another aspect, the back portion can be configured to surround at least a portion of the back of the wearer. In another aspect, the chest and back portions of the harness can cooperate to define opposed first and second openings that can be configured to receive respective arms of the wearer.

In yet another aspect, the chest portion of the harness can comprise first and second attachment zones. In another aspect, a first neck strap of the pair of neck straps can be configured for selective secure attachment to the first attachment zone of the chest portion of the harness. In a further aspect, a second neck strap of the pair of neck straps can be configured for selective secure attachment to the second attachment zone of the chest portion of the harness.

Also disclosed herein are methods for supporting a head and neck of a wearer. In one aspect, the method can comprise positioning a head cover, as described herein, on the head of the wearer. In another aspect, the method can comprise positioning a harness, as described herein, in an operative position on the wearer. In still another aspect, the method can comprise selectively securely attaching a first neck strap of the pair of neck straps to the first attachment zone of the chest portion of the harness. In yet another aspect, the method can comprise selectively securely attaching a second neck strap of the pair of neck straps to the second attachment zone of the chest portion of the harness.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION OF THE FIGURES

The accompanying drawings, which are incorporated in and constitute a part of this specification, illustrate several aspects of the invention and together with the description, serve to explain the principles of the invention.

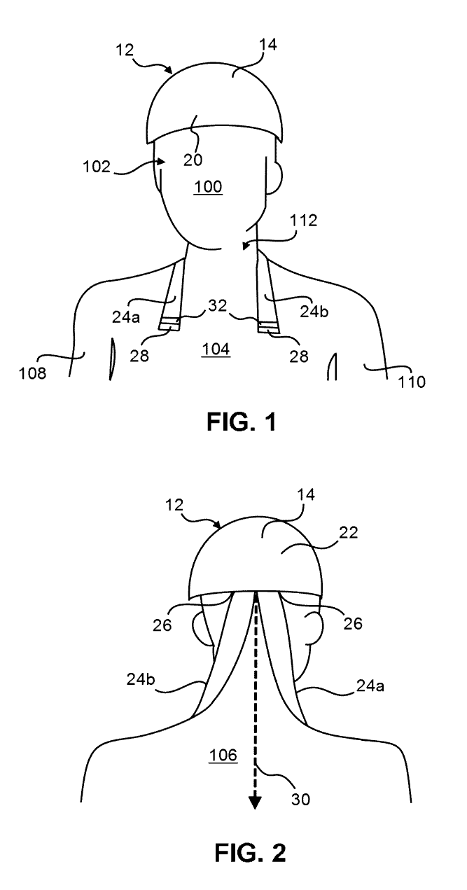

FIG. 1 is a front view of a head cover of an exemplary head and neck support device as disclosed herein.

FIG. 2 is a rear view of the head cover of FIG. 1.

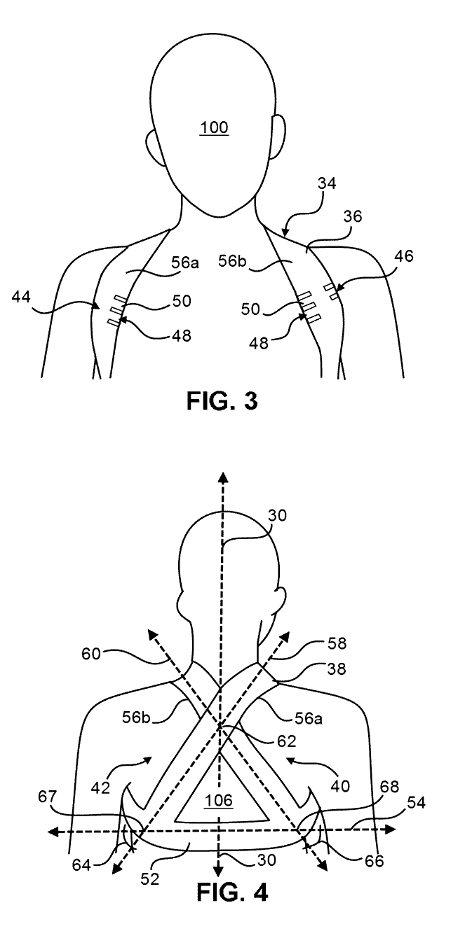

FIG. 3 is a front view of a harness of an exemplary head and neck support device as disclosed herein.

FIG. 4 is a rear view of the harness of FIG. 3.

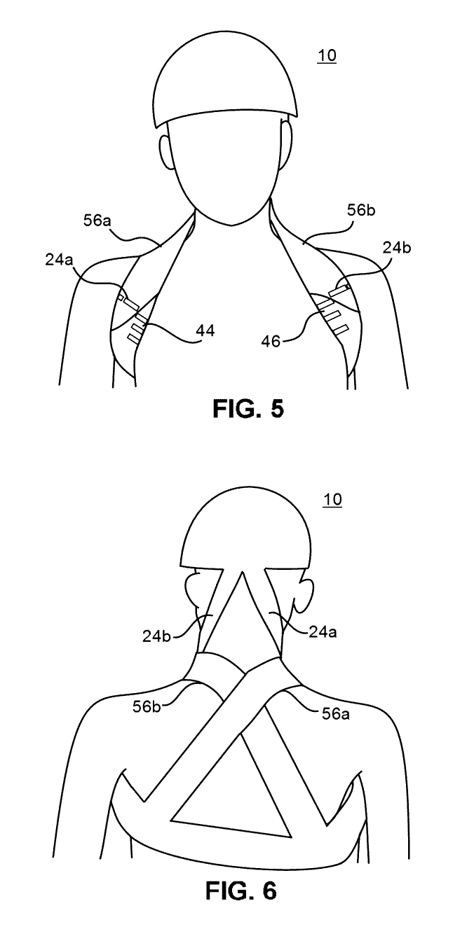

FIG. 5 is a front view of an exemplary head and neck support device as disclosed herein.

FIG. 6 is a rear view of the head and neck support device of FIG. 5.

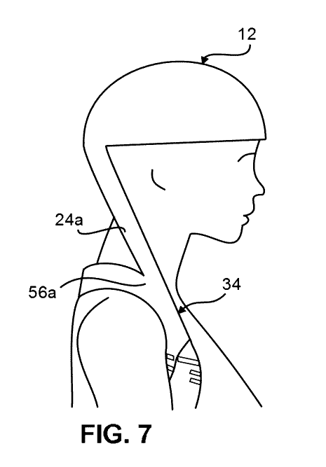

FIG. 7 is a right side view of the head and neck support device of FIG. 5.

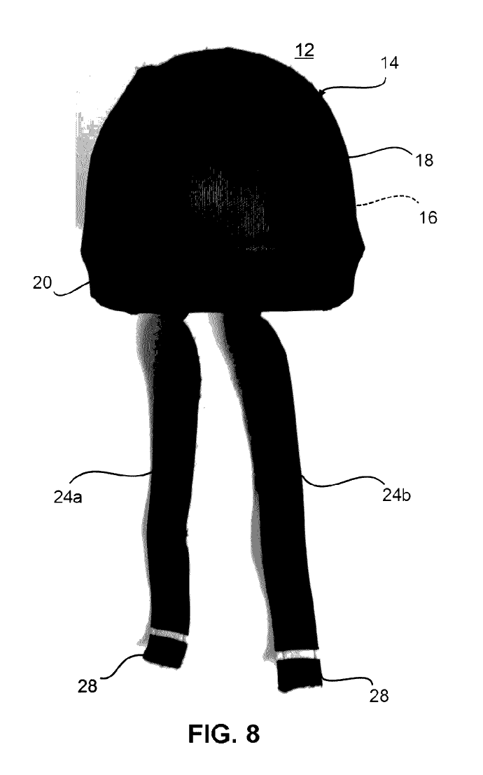

FIG. 8 is a front view of an exemplary head cover as disclosed herein.

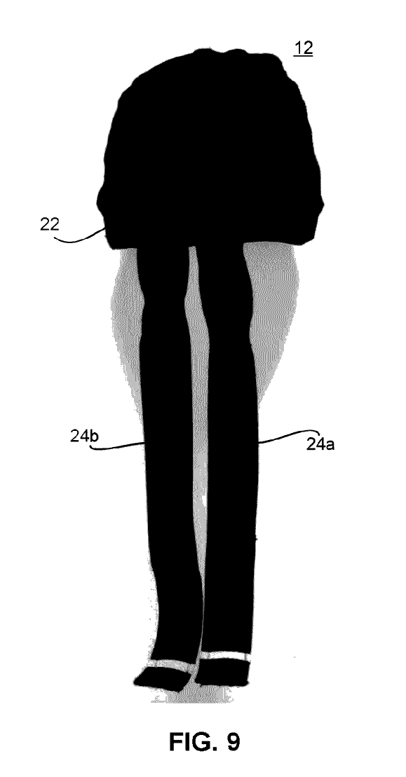

FIG. 9 is a rear view of the head cover of FIG. 8.

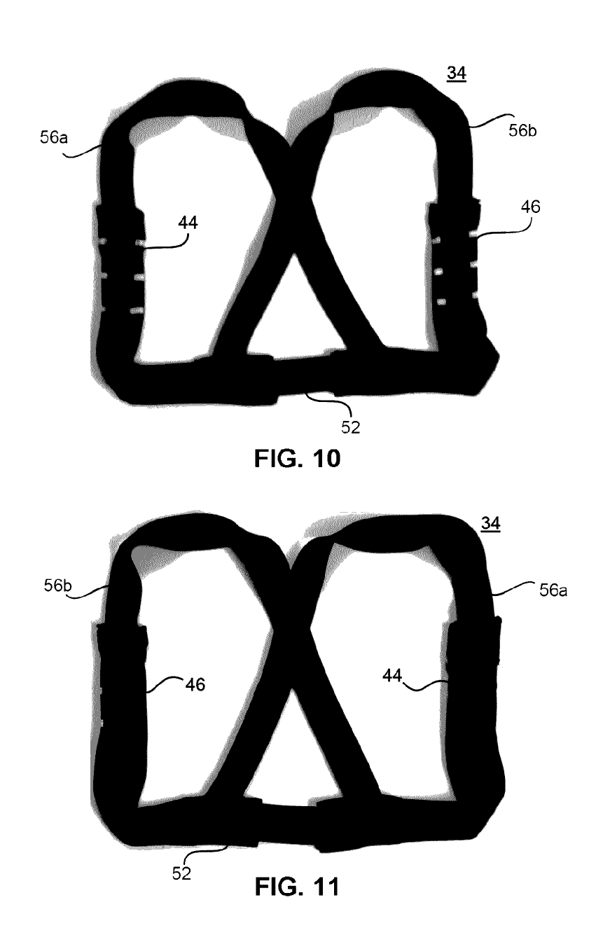

FIG. 10 is a front view of an exemplary harness as disclosed herein.

FIG. 11 is a rear view of the harness of FIG. 10.

### DETAILED DESCRIPTION

The present invention can be understood more readily by reference to the following detailed description, examples, drawings, and claims, and their previous and following descriptions. However, before the present devices and/or methods are disclosed and described, it is to be understood that this invention is not limited to the specific devices and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used throughout, the singular forms “a,” “an” and “the” include plural referents unless the context clearly dictates otherwise. Thus, for example, reference to “a fastener” can include two or more such fasteners unless the context indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

The word “or” as used herein means any one member of a particular list and also includes any combination of members of that list.

The terms “first,” “second,” “first part,” “second part,” and the like, as used herein, do not denote any order, quantity, or importance, and are used to distinguish one element from another, unless specifically stated otherwise.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

Referring now to FIGS. 1-11, disclosed herein is a head and neck support device 10. The head and neck support device 10 can be used to support weakened muscles of the neck due to aging, poor posture, and/or injury. In one aspect, as shown in FIGS. 1-2 and 5-9, the head and neck support device 10 can comprise a head cover 12. In this aspect, the head cover 12 can comprise a head-receiving component 14 that has an interior surface 16 and an exterior surface 18. In a further aspect, the head-receiving component 14 can have a front portion 20 and an opposed rear portion 22. In another aspect, it is contemplated that the interior surface 16 of the head-receiving component 14 can be configured to receive at least a portion of the head 102 of a wearer 100. It is further contemplated that the head-receiving component 14 can conform or generally conform to the contour of the wearer's head 102. In one aspect, the head cover 12, including the head-receiving component 14, can be made of a light, slightly elastic material to fit different head shapes and sizes. Such light, slightly elastic materials are well known in the art, although some specific examples are provided herein.

In another aspect, as shown in FIGS. 1-2 and 8-9, the head cover 12 can comprise a plurality of neck straps (optionally, a pair of neck straps 24a, 24b). In one aspect, the head and neck support device 10 can comprise a first neck strap 24a and a second neck strap 24b. In this aspect, each neck strap 24a, 24b can have a first end 26 secured to the rear portion 22 of the head-receiving component and an opposed second end 28. In another aspect, it is contemplated that each neck strap 24a, 24b can extend downwardly from the head-receiving component 14 relative to a vertical axis 30 (or another axis that, in use, is parallel or substantially parallel to the spine of the wearer). It is contemplated that the second ends 28 of each of the first and second neck straps 24a, 24b can comprise a fastener 32 or group of fasteners. In one aspect, the fastener 32 can be configured to secure the neck strap 24 to a respective attachment zone 44, 46 of a chest portion 36 of a harness 34 as further disclosed herein. The fastener 32 of the second end 28 of each neck strap 24a, 24b can be selected from the group consisting of a clamp, a buckle, a latch, a clip, an eye and hook, hook and loop fasteners, a snap button, a press stud, a button, a zipper, and combinations thereof. However, it is contemplated that any conventional fastening mechanism can be used.

In another aspect, as shown in FIGS. 3-4 and 10-11, the head and neck support device 10 can comprise a harness 34. In one aspect, the harness 34 can have a chest portion 36 and an opposed back portion 38. In another aspect, the chest portion 36 can be configured to surround at least a portion of the chest 104 of the wearer 100. In another aspect, the back portion 38 can be configured to surround at least a portion of the back 106 of the wearer 100. In another aspect, the chest and back portions 36, 38 of the harness 34 can cooperate to define opposed first and second openings 40, 42 that can be configured to receive respective arms of the wearer 108, 110. More particularly, after both arms of a wearer are passed through the first and second openings 40, 42, the harness covers portions of the chest, back and shoulders of the wearer while leaving at least a portion of both arms (e.g. substantially the entirety of both arms) of the wearer exposed. In some optional aspects, it is contemplated that the harness 34 can be provided as a vest, which can include chest panels that extend outwardly from shoulder straps to cover additional portions of the chest of the wearer. However, as shown in the Figures, it is contemplated that the chest portion 36 of the harness need only include a front/chest portion of the shoulder straps.

In yet another aspect, as shown in FIGS. 3, 5, and 10, the chest portion 36 of the harness 34 can comprise first and second attachment zones 44, 46. In another aspect, the first neck strap 24a of the pair of neck straps can be configured for selective secure attachment to the first attachment zone 44 of the chest portion 36 of the harness 34. In a further aspect, the second neck strap 24b of the pair of neck straps can be configured for selective secure attachment to the second attachment zone 46 of the chest portion 36 of the harness 34. In one aspect, it is contemplated that each of the first and second attachment zones 44, 46 of the chest portion 36 of the harness 34 can comprise a fastener 48 or group of fasteners 48. In another aspect, the fastener 48 can be configured to secure the chest portion 36 of the harness 34 to a respective neck strap 24a, 24b of the pair of neck straps. In this aspect, it is contemplated that fastener 32 can be complementary to fastener 48 to ensure a secure connection between the chest portion 36 and the neck straps 24a, 24b. In another aspect, it is further contemplated that the fastener 48 of each of the first and second attachment zones 44, 46 can be selected from the group consisting of a clamp, a buckle, a latch, a clip, an eye and hook, hook and loop fasteners, a snap button, a press stud, a button, a zipper, and combinations thereof. However, it is contemplated that any conventional fastening mechanism can be used. In another aspect, the first and second attachment zones 44, 46 can comprise respective pads 50 that can be sewn or otherwise secured to the chest portion 36 of the harness 36. In exemplary non-limiting aspects, it is contemplated that the fastener 32 of the second end 28 of each neck strap 24a, 24b and the fastener 48 of each of the first and second attachment zones 44, 46 can comprise complementary hook and loop fasteners.

Optionally, in another aspect, as shown in FIGS. 4, 6, and 11, the back portion 38 of the harness 34 can comprise at least one back supporting strap 52. Optionally, the at least one back supporting strap 52 can comprise a single back supporting strap. Alternatively, the at least one back supporting strap 52 can comprise a plurality of back supporting straps. Optionally, a plurality of back supporting straps can be spaced relative to the vertical axis 30 (or other axis parallel or substantially parallel to the spine of the wearer). Optionally, in an exemplary aspect, at least one back supporting strap 52 can extend perpendicularly or substantially perpendicularly to the vertical axis 30. In a further aspect, the harness 34 can comprise a plurality of shoulder straps (optionally, a pair of shoulder straps 56a, 56b), as shown in FIGS. 3, 4, 6, and 7. The shoulder straps 56a, 56b can be coupled to the at least one back supporting strap 52. Optionally, it is contemplated that the operative lengths of the shoulder straps 56a, 56b and/or the at least one back supporting strap 52 can be selectively adjustable to fit various shapes and sizes of wearers and also to allow the wearer to adjust the straps throughout his or her use of the device 10. In these aspects, the at least one back supporting strap 52 and the shoulder straps 56a, 56b can cooperate to define the first and second openings 40, 42 of the harness 34. In another aspect, the first and second openings 40, 42 of the harness 34 can have a round or substantially round (e.g., oval, substantially oval, elliptical, or substantially elliptical) shape. In still another aspect, at least a portion of each of a first and second shoulder strap 56a, 56b can have an arcuate profile that defines a corresponding portion of the first and second openings 40, 42.

In another aspect, a first shoulder strap 56a of the pair of shoulder straps can overlie at least a portion of a second shoulder strap 56b of the pair of shoulder straps. Alternatively, in another aspect, a second shoulder strap 56b of the pair of shoulder straps 56 can overlie at least a portion of a first shoulder strap 56a of the pair of shoulder straps 56. In these aspects, regardless of whether the first shoulder strap 56a overlies the second shoulder strap 56b or the second shoulder strap 56b overlies the first shoulder strap 56a, the first and second shoulder straps 56a, 56b can have respective first and second longitudinal axes 58, 60 that extend away from the at least one back supporting strap 52. It is contemplated, in one aspect, that the first longitudinal axis 58 can intersect the second longitudinal axis 60 at an intersection point 62. In an operative position on the wearer 100, the intersection point 62 can be configured to be in substantial alignment with the spine of the wearer 100. Thus, it is contemplated that that the intersection point can correspond to a midline position relative to an operative width of the device (measured relative to an axis that is perpendicular or substantially perpendicular to the vertical axis or another axis aligned with the spine of the wearer).

Optionally, in exemplary aspects, rather than overlying one another (with at least one overlapping section with multiple layers), it is contemplated that the first and second shoulder straps 56a, 56b can be secured to one another and/or integrally formed in an X-pattern as shown in FIG. 4. In some aspects, when the first and second shoulder straps 56a, 56b are formed in an X-pattern, it is contemplated that the first and second shoulder straps can be secured together in a single layer.

In another aspect, the at least one back supporting strap 52 can comprise a first back supporting strap having a longitudinal axis 54. In one aspect, it is contemplated that the distance between intersection point 62 and the point at which the longitudinal axis 54 of the first back supporting strap 52 and the vertical axis 30 intersect can range from about 4 inches to about 30 inches, from about 4.5 inches to about 20 inches, or from about 5 inches to about 11 inches. In one aspect, it is contemplated that the distance between intersection point 62 and the point at which the longitudinal axis 54 of the first back supporting strap 52 and the vertical axis 30 intersect can range from about 8 inches to about 12 inches or from about 10.0 inches to about 10.5 inches. In another aspect, the distance between intersection point 62 and the point at which the longitudinal axis 54 of the first back supporting strap 52 and the vertical axis 30 intersect can range from about 6 inches to about 10 inches or from about 7.5 inches to about 8.0 inches. In still another aspect, the distance between intersection point 62 and the point at which the longitudinal axis 54 of the first back supporting strap 52 and the vertical axis 30 intersect can range from about 4 inches to about 8 inches or from about 5.5 inches to about 6.0 inches. Although specific exemplary dimensions are provided above, it is contemplated that the disclosed distances can be any desired distances that allow the device to function as described herein.

In another aspect, the first and second shoulder straps 56a, 56b can have respective first and second longitudinal axes 58, 60 that extend away from first back supporting strap 52. In a further aspect, the longitudinal axes 58, 60 of the first and second shoulder straps 56a, 56b can be positioned at respective first and second acute angles 64, 66 relative to the longitudinal axis 54 of the first back supporting strap 52. In still another aspect, the first and second acute angles 64, 66 can range from about 30 degrees to about 75 degrees. In another aspect, as shown in FIG. 4, the pair of shoulder straps 56a, 56b and a first back supporting strap 52a of the at least one back supporting strap 52 can cooperate to form an equilateral or substantially equilateral triangle. In one aspect, the distance between the two intersection points 67, 68 at which the first and second longitudinal axes 58, 60 intersect with the longitudinal axis 54 of the first back supporting strap can range from about 2 inches to about 20 inches, from about 2.5 inches to about 15 inches, or from about 3.0 inches to about 8.0 inches. In another aspect, the distance between the two intersection points at which the first and second longitudinal axes 58, 60 intersect with the longitudinal axis 54 of the first back supporting strap can range from about 7.5 inches to about 8.0 inches. In still another aspect, the distance between the two intersection points at which the first and second longitudinal axes 58, 60 intersect with the longitudinal axis 54 of the first back supporting strap can range from about 4 inches to about 7 inches or from about 5.0 inches to about 5.5 inches. In yet another aspect, the distance between the two intersection points at which the first and second longitudinal axes 58, 60 intersect with the longitudinal axis 54 of the first back supporting strap can range from about 2 inches to about 5 inches or from about 3.0 inches to about 3.5 inches. Although specific exemplary dimensions are provided above, it is contemplated that the disclosed distances can be any desired distances that allow the device to function as described herein.

In exemplary aspects, it is contemplated that the longitudinal axes 54, 58, 60 of the first back supporting strap 52 and the first and second shoulder straps 56a, 56b can be straight or substantially straight. However, in other optional aspects, it is contemplated that at least one of the back supporting strap 52 and the shoulder straps 56a, 56b can have an arcuate or other non-linear profile such that the longitudinal axes 54, 58, 60 correspond to central axes that are positioned centrally between the opposing side edges of the straps along the respective lengths of the straps.

In another aspect, it is contemplated that at least a portion of the head and neck support device 10 (e.g., at least a portion of the head-receiving component, the harness, the back supporting straps, and/or shoulder straps) can be flexible, elastic, and/or resilient. In another aspect, at least a portion of the head and neck support device 10 (e.g., at least a portion of the head-receiving component, the harness, the back supporting straps, and/or shoulder straps) can comprise one or more of SPANDEX, polyester, nylon, rayon, polyactide, fiber, olefin fiber, aramid fiber, an acrylic, ingeo, lurex, cotton, leather, rubber, and combinations thereof.

In another aspect, the head and neck support device 10 can be configured to increase a range of motion of the head 102 of the wearer 100 in at least one direction. In another aspect, the head and neck support device 10 can be configured to maintain the head 102 of the wearer 100 in an upright position. In yet a further aspect, in use, the head and neck support device 100 can be configured to apply a tensile force that opposes a natural downward movement of the head 102 of the wearer 100.

In use, the head and neck support device 10 disclosed herein can be used in a method for supporting a head 102 and neck 112 of a wearer 100. In one aspect, the method can comprise positioning a head cover 12 on the head 102 of the wearer 100. In another aspect, the method can comprise positioning a harness 34 in an operative position (e.g., over the chest and back) on the wearer 100. Following positioning of the harness 34 in the operative position, it is contemplated that the shoulder straps or the back supporting straps (when present) can be selectively adjusted to obtain a desired fit over the wearer. In another aspect, a first neck strap 24a of the pair of neck straps 24 can be selectively securely attached to the first attachment zone 44 of the chest portion 36 of the harness 34. In a further aspect, a second neck strap 24b of the pair of neck straps 24 can be selectively securely attached to the second attachment zone 46 of the chest portion 36 of the harness 34. It is further contemplated that, during initial positioning of the head cover 12, the neck straps can be selectively pulled to form a sufficiently tight fit over at least a portion of the head of the wearer. Following attachment of the first and second neck straps 24a, 24b to the first and second attachment zones 44, 46, in one aspect, the head cover 12 and the harness 34 can increase a range of motion of the head 102 of the wearer 100 in at least one direction (e.g., left, right, up, or down). Optionally, the head cover 12 and the harness 34 can increase a range of motion of the head 102 of the wearer 100 in a plurality of directions (e.g., at least two of left, right, up, and down). In another aspect, the head cover 12 and the harness 34 can maintain the head 102 of the wearer 100 in an upright position. In a further aspect, the head cover 12 and the harness 34 can cooperate to apply a tensile force that opposes a natural downward movement of the head 102 of the wearer 100. After use of the device, the neck straps can be removed from the first and second attachment zones to thereby detach the head cover 12 from the harness 34. With the head cover detached from the harness, the shoulder straps and/or back supporting straps (when present) can be selectively loosened to permit removal of the harness from the wearer. Alternatively, if the harness is provided as a vest, it is contemplated that the method can comprise removing the vest from the wearer.

### Exemplary Aspects

In view of the described devices and methods and variations thereof, herein below are described certain more particularly described aspects of the invention. These particularly recited aspects should not, however, be interpreted to have any limiting effect on any different claims containing different or more general teachings described herein, or that the “particular” aspects are somehow limited in some way other than the inherent meanings of the language literally used therein.

Aspect 1: A head and neck support device, comprising: a head cover comprising: a head-receiving component having an interior surface, an exterior surface, a front portion, and an opposed rear portion, wherein the interior surface of the head-receiving component is configured to receive at least a portion of the head of a wearer; and a pair of neck straps, wherein each neck strap has a first end secured to the rear portion of the hand-receiving portion and an opposed second end, and wherein each neck strap extends downwardly from the head-receiving component relative to a vertical axis; a harness having a chest portion and an opposed back portion, wherein the chest portion is configured to surround at least a portion of the chest of the wearer, wherein the back portion is configured to surround at least a portion of the back of the wearer, wherein the chest and back portions of the harness cooperate to define opposed first and second openings that are configured to receive respective arms of the wearer, and wherein the chest portion of the harness comprises first and second attachment zones, wherein a first neck strap of the pair of neck straps is configured for selective secure attachment to the first attachment zone of the chest portion of the harness, and wherein a second neck strap of the pair of neck straps is configured for selective secure attachment to the second attachment zone of the chest portion of the harness.

Aspect 2: The head and neck support device of aspect 1, wherein the harness comprises at least one back supporting strap extending perpendicularly or substantially perpendicularly to the vertical axis.

Aspect 3: The head and neck support device of any one of the preceding aspects, wherein the harness further comprises a pair of shoulder straps coupled to the at least one back supporting strap, wherein the at least one back supporting strap and the pair of shoulder straps cooperate to define the first and second openings of the harness.

Aspect 4: The head and neck support device of any one of the preceding aspects, wherein the head and neck support device is configured to increase a range of motion of the head of the wearer in at least one direction.

Aspect 5: The head and neck support device of any one of the preceding aspects, wherein the head and neck support device is configured to maintain the head of the wearer in an upright position.

Aspect 6: The head and neck support device of any one of the preceding aspects, wherein the head and neck support device is configured to apply a tensile force that opposes a natural downward movement of the head of the wearer.

Aspect 7: The head and neck support device of any one of the preceding aspects, wherein at least a portion of the head and neck support device is flexible, elastic, and resilient.

Aspect 8: The head and neck support device of any one of the preceding aspects, wherein at least a portion of the head and neck support device comprises one or more of SPANDEX, polyester, nylon, rayon, polylactide fiber, olefin fiber, aramid fiber, an acrylic, ingeo, lurex, cotton, leather, rubber, and combinations thereof.

Aspect 9: The head and neck support device of any one of the preceding aspects, wherein the second ends of each of the first and second neck straps comprises a fastener that is configured to secure the neck strap to a respective attachment zone of the chest portion of the harness.

Aspect 10: The head and neck support device of any one of the preceding aspects, wherein the fastener of the second end of each neck strap is selected from the group consisting of a clamp, a buckle, a latch, a clip, an eye and hook, hook and loop fasteners, a snap button, a press stud, a button, a zipper, and combinations thereof.

Aspect 11: The head and neck support device of any one of the preceding aspects, wherein each of the first and second attachment zones of the chest portion of the harness comprises a fastener that is configured to secure the chest portion of the harness to a respective neck strap of the pair of neck straps.

Aspect 12: The head and neck support device of any one of the preceding aspects, wherein the fastener of each of the first and second attachment zones is selected from the group consisting of a clamp, a buckle, a latch, a clip, an eye and hook, hook and loop fasteners, a snap button, a press stud, a button, a zipper, and combinations thereof.

Aspect 13: The head and neck support device of any one of the preceding aspects, wherein the second ends of each of the first and second neck straps comprises a fastener, and wherein each of the first and second attachment zones of the chest portion comprises a fastener that is configured for engagement with the fastener of the second end of a corresponding neck strap.

Aspect 14: The head and neck support device of any one of the preceding aspects, wherein the fasteners of the second end of each neck strap and the first and second attachment zones are respectively selected from the group consisting of a clamp, a buckle, a latch, a clip, an eye and hook, hook and loop fasteners, a snap button, a press stud, a button, a zipper, and combinations thereof.

Aspect 15: The head and neck support device of any one of the preceding aspects, wherein the first and second attachment zones comprise respective pads that are sewn to the chest portion of the harness.

Aspect 16: The head and neck support device of any one of the preceding aspects, wherein the operative lengths of the pair of shoulder straps and the at least one back supporting strap are selectively adjustable.

Aspect 17: The head and neck support device of any one of the preceding aspects, wherein a first shoulder strap of the pair of shoulder straps overlies at least a portion of a second shoulder strap of the pair of shoulder straps, and wherein the first and second shoulder straps have respective first and second longitudinal axes that extend away from the at least one back supporting strap.

Aspect 18: The head and neck support device of any one of the preceding aspects, wherein a second shoulder strap of the pair of shoulder straps overlies at least a portion of a first shoulder strap of the pair of shoulder straps, and wherein the first and second shoulder straps have respective first and second longitudinal axes that extend away from the at least one back supporting strap.

Aspect 19: The head and neck support device of any one of the preceding aspects, wherein the first longitudinal axis intersects the second longitudinal axis at an intersection point, and wherein in an operative position on the wearer, the intersection point is configured to be in substantial alignment with the spine of the wearer.

Aspect 20: The head and neck support device of any one of the preceding aspects, wherein the first longitudinal axis intersects the second longitudinal axis at an intersection point, and wherein in an operative position on the wearer, the intersection point is configured to be in substantial alignment with the spine of the wearer.

Aspect 21: The head and neck support device of any one of the preceding aspects, wherein the pair of shoulder straps and a first back supporting strap of the at least one back supporting strap cooperate to form an equilateral or a substantially equilateral triangle.

Aspect 22: The head and neck support device of any one of the preceding aspects, wherein the at least one back supporting strap comprises a first back supporting strap having a longitudinal axis that is perpendicular or substantially perpendicular to the vertical axis, wherein the first and second shoulder straps have respective first and second longitudinal axes that extend away from the first back supporting strap, and wherein the longitudinal axes of the first and second shoulder straps are positioned at respective first and second acute angles relative to the longitudinal axis of the first back supporting strap.

Aspect 23: The head and neck support device of any one of the preceding aspects, wherein the first and second acute angles range from about 30 degrees to about 75 degrees.

Aspect 24: The head and neck support device of any one of the preceding aspects, wherein at least a portion of each of the first and second shoulder straps has an arcuate profile that defines a corresponding portion of the first and second openings.

Aspect 25: A method for supporting a head and neck of a wearer, comprising, positioning a head cover on the head of the wearer, wherein the head cover comprises: a head-receiving component having an interior surface, an exterior surface, a front portion, and an opposed rear portion, wherein the interior surface of the head cover receives at least a portion of the head of a wearer; and a pair of neck straps, wherein each neck strap has a first end secured to the rear portion and an opposed second end, and wherein each neck strap extends downwardly from the head-receiving component relative to a vertical axis; positioning a harness in an operative position on the wearer, wherein the harness comprises a chest portion and an opposed back portion, wherein in the operative position the chest portion surrounds at least a portion of the chest of the wearer, wherein the back portion surrounds at least a portion of the back of the wearer, wherein the chest and back portions of the harness cooperate to define opposed first and second openings that, in the operative position, receive respective arms of the wearer, and wherein the chest portion of the harness comprises first and second attachment zones; selectively securely attaching a first neck strap of the pair of neck straps to the first attachment zone of the chest portion of the harness; and selectively securely attaching a second neck strap of the pair of neck straps to the second attachment zone of the chest portion of the harness.

Aspect 26: The method of aspect 25, wherein following attachment of the first and second neck straps to the first and second attachment zones, the head cover and the harness increase a range of motion of the head of the wearer in at least one direction.

Aspect 27: The method of any one of the preceding aspects, wherein following attachment of the first and second neck straps to the first and second attachment zones, the head cover and the harness maintain the head of the wearer in an upright position.

Aspect 28: The method of any one of the preceding aspects, wherein following attachment of the first and second neck straps to the first and second attachment zones, the head cover and the harness cooperate to apply a tensile force that opposes a natural downward movement of the head of the wearer.

Aspect 29: The method of any one of the preceding aspects, wherein the harness comprises at least one back supporting strap extending perpendicularly or substantially perpendicularly to the vertical axis, wherein the harness further comprises a pair of shoulder straps coupled to the at least one back supporting strap, wherein the at least one back supporting strap and the pair of shoulder straps cooperate to define the first and second openings of the harness, wherein a first shoulder strap of the pair of shoulder straps overlies at least a portion of a second shoulder strap of the pair of shoulder straps, and wherein the first and second shoulder straps have respective first and second longitudinal axes that extend away from the at least one back supporting strap.

Aspect 30: The method of any one of the preceding aspects, wherein the first longitudinal axis intersects the second longitudinal axis at an intersection point, and wherein in an operative position on the wearer, the intersection point is in substantial alignment with the spine of the wearer.

Although several embodiments of the invention have been disclosed in the foregoing specification, it is understood by those skilled in the art that many modifications and other embodiments of the invention will come to mind to which the invention pertains, having the benefit of the teaching presented in the foregoing description and associated drawings. It is thus understood that the invention is not limited to the specific embodiments disclosed hereinabove, and that many modifications and other embodiments are intended to be included within the scope of the appended claims. Moreover, although specific terms are employed herein, as well as in the claims which follow, they are used only in a generic and descriptive sense, and not for the purposes of limiting the described invention, nor the claims which follow.

What is claimed is:

1. A head and neck support device, comprising:
a head cover comprising:
a head-receiving component having an interior surface, an exterior surface, a front portion, and an opposed rear portion, wherein the interior surface of the head-receiving component is configured to receive at least a portion of the head of a wearer; and
a pair of neck straps, wherein each neck strap has a first end secured to the rear portion of the hand-receiving portion and an opposed second end, and wherein each neck strap extends downwardly from the head-receiving component relative to a vertical axis; and
a harness having a chest portion and an opposed back portion, wherein the chest portion is configured to surround at least a portion of the chest of the wearer, wherein the back portion is configured to surround at least a portion of the back of the wearer, wherein the chest and back portions of the harness cooperate to define opposed first and second openings that are configured to receive respective arms of the wearer, and wherein the chest portion of the harness comprises first and second attachment zones,
wherein a first neck strap of the pair of neck straps is configured for selective secure attachment to the first attachment zone of the chest portion of the harness, and wherein a second neck strap of the pair of neck straps is configured for selective secure attachment to the second attachment zone of the chest portion of the harness.

2. The head and neck support device of claim 1, wherein the harness comprises at least one back supporting strap extending substantially perpendicularly to the vertical axis.

3. The head and neck support device of claim 2, wherein the harness further comprises a pair of shoulder straps coupled to the at least one back supporting strap, wherein the at least one back supporting strap and the pair of shoulder straps cooperate to define the first and second openings of the harness.

4. The head and neck support device of claim 1, wherein at least a portion of the head and neck support device is flexible, elastic, and resilient.

5. The head and neck support device of claim 1, wherein at least a portion of the head and neck support device comprises one or more of SPANDEX, polyester, nylon, rayon, polylactide fiber, olefin fiber, aramid fiber, an acrylic, ingeo, lurex, cotton, leather, rubber, and combinations thereof.

6. The head and neck support device of claim 1, wherein the second ends of each of the first and second neck straps comprises a fastener that is configured to secure the neck strap to a respective attachment zone of the chest portion of the harness.

7. The head and neck support device of claim 1, wherein each of the first and second attachment zones of the chest portion of the harness comprises a fastener that is configured to secure the chest portion of the harness to a respective neck strap of the pair of neck straps.

8. The head and neck support device of claim 1, wherein the second ends of each of the first and second neck straps comprises a fastener, and wherein each of the first and second attachment zones of the chest portion comprises a fastener that is configured for engagement with the fastener of the second end of a corresponding neck strap.

9. The head and neck support device of claim 1, wherein the first and second attachment zones comprise respective pads that are sewn to the chest portion of the harness.

10. The head and neck support device of claim 3, wherein the operative lengths of the pair of shoulder straps and the at least one back supporting strap are selectively adjustable.

11. The head and neck support device of claim 3, wherein a first shoulder strap of the pair of shoulder straps overlies at least a portion of a second shoulder strap of the pair of shoulder straps, and wherein the first and second shoulder straps have respective first and second longitudinal axes that extend away from the at least one back supporting strap.

12. The head and neck support device of claim 3, wherein the pair of shoulder straps and a first back supporting strap of the at least one back supporting strap cooperate to form a substantially equilateral triangle.

13. The head and neck support device of claim 3, wherein the at least one back supporting strap comprises a first back supporting strap having a longitudinal axis that is substantially perpendicular to the vertical axis, wherein the first and second shoulder straps have respective first and second longitudinal axes that extend away from the first back supporting strap, and wherein the longitudinal axes of the first and second shoulder straps are positioned at respective first and second acute angles relative to the longitudinal axis of the first back supporting strap.

14. The head and neck support device of claim 13, wherein the first and second acute angles range from about 30 degrees to about 75 degrees.

15. The head and neck support device of claim 3, wherein at least a portion of each of the first and second shoulder straps has an arcuate profile that defines a corresponding portion of the first and second openings.

16. A method for supporting a head and neck of a wearer, comprising,
positioning a head cover on the head of the wearer, wherein the head cover comprises:
a head-receiving component having an interior surface, an exterior surface, a front portion, and an opposed rear portion, wherein the interior surface of the head cover receives at least a portion of the head of a wearer; and
a pair of neck straps, wherein each neck strap has a first end secured to the rear portion and an opposed second end, and wherein each neck strap extends downwardly from the head-receiving component relative to a vertical axis;
positioning a harness in an operative position on the wearer, wherein the harness comprises a chest portion and an opposed back portion, wherein in the operative position the chest portion surrounds at least a portion of the chest of the wearer, wherein the back portion surrounds at least a portion of the back of the wearer, wherein the chest and back portions of the harness cooperate to define opposed first and second openings that, in the operative position, receive respective arms of the wearer, and wherein the chest portion of the harness comprises first and second attachment zones;
selectively securely attaching a first neck strap of the pair of neck straps to the first attachment zone of the chest portion of the harness; and
selectively securely attaching a second neck strap of the pair of neck straps to the second attachment zone of the chest portion of the harness.

17. The method of claim 16, wherein following attachment of the first and second neck straps to the first and second attachment zones, the head cover and the harness maintain the head of the wearer in an upright position.

18. The method of claim 17, wherein following attachment of the first and second neck straps to the first and second attachment zones, the head cover and the harness cooperate to apply a tensile force that opposes a natural downward movement of the head of the wearer.

19. The method of claim 16, wherein the harness comprises at least one back supporting strap extending substantially perpendicularly to the vertical axis, wherein the harness further comprises a pair of shoulder straps coupled to the at least one back supporting strap, wherein the at least one back supporting strap and the pair of shoulder straps cooperate to define the first and second openings of the harness, wherein a first shoulder strap of the pair of shoulder straps overlies at least a portion of a second shoulder strap of the pair of shoulder straps, and wherein the first and second shoulder straps have respective first and second longitudinal axes that extend away from the at least one back supporting strap.

20. The method of claim 19, wherein the first longitudinal axis intersects the second longitudinal axis at an intersection point, and wherein in an operative position on the wearer, the intersection point is in substantial alignment with the spine of the wearer.
